(19) United States
(12) Patent Application Publication
(10) Pub. No.: US 2019/0339256 A1
(43) Pub. Date: Nov. 7, 2019

(54) METHOD FOR SCREENING REFRESHING ESSENTIAL OILS OR PRODUCTS THEREOF

(71) Applicant: INFINITUS (CHINA) COMPANY LTD., Jiang Men, Guangdong (CN)

(72) Inventors: Lei XIAO, Jiang Men, Guangdong (CN); Yu CHEN, Jiang Men, Guangdong (CN); Jinlian HUANG, Jiang Men, Guangdong (CN)

(73) Assignee: INFINITUS (CHINA) COMPANY LTD., Jiang Men, Guangdong (CN)

(21) Appl. No.: 16/467,478

(22) PCT Filed: Jun. 23, 2017
(86) PCT No.: PCT/CN2017/089697

§ 371 Date: Jun. 6, 2019

(30) Foreign Application Priority Data
May 27, 2017 (CN) 201710391002.1

(51) Int. Cl. G01N 33/50

(52) U.S. Cl. CPC G01N 33/5058

(57) ABSTRACT

Provided by the present application is a method for screening refreshing essential oils or products thereof, comprising: processing cerebral nerve cells using essential oils or products thereof; detecting the concentration of neurotransmitters such as 5-hydroxy tryptamine; obtaining a refreshing score for each neurotransmitter by means of the change in the concentrations of a treatment group and a control group; and calculating the total refreshing score and using the same to screen refreshing essential oils or products thereof.

### CROSS REFERENCE TO RELATED APPLICATIONS

This application claims the priority of Chinese Patent Application No. 201710391002.1, filed on May 27, 2017, and titled with “METHOD FOR SCREENING REFRESHING ESSENTIAL OILS OR PRODUCTS THEREOF”, and the disclosures of which are hereby incorporated by reference.

### FIELD

The present disclosure relates to the field of daily chemical technology, specifically to a method for screening essential oils with a refreshing efficacy or their products.

### BACKGROUND

With the development of society, the pace of modern life is accelerating, the competition for survival is becoming more and more fierce, and the pressure of work and study is increasing. Especially for business people, white-collar workers and students, they often feel tired and dizzy. Some people are prone to feel sleepy in spring, hot summers and autumns. Especially for drivers, they need to be refreshed and avoid danger when they are tired and sleepy. As a result, consumers' demand for products with a refreshing efficacy is increasing.

Essential oils are volatile aromatic substances extracted from flowers, leaves, stems, roots or fruits of plants by steam distillation method, extrusion method, cold-maceration method or solvent extraction method. Aromatic essential oils have a function on the central nervous system of the human body. This effect is due to the effects of olfactory-sensing neuron cells and their receptors on brain function. Such functions include behaviors such as refreshment and alertness. Essential oils are made up of some small molecules, and are high. volatile substances which can be absorbed into the body by nasal mucosal tissue, send messages directly to the brain, and regulate the emotion and physical functions of the body through the limbic system of the brain. Essential oils have the functions of stimulating sympathetic nerve and parasympathetic nerve, sedation and hypnosis, refreshing, regulating mental status, anti-depression, relieving psychological stress, and repairing the nervous system. Animal studies have shown that certain aromatic essential oils may increase the concentration of dopamine in body fluids to some extent.

At present, the evaluation of the efficacies of aromatic essential oils is usually based on species of the raw material, origin of the raw material, composition of the raw material, content of the raw material, extraction and preparation process of the raw material, physical properties of the raw material including freezing point, melting point, boiling point, density, refractive index and color, smell of the raw material, traditional experience in the use of the raw material, as well as a small amount of using experience of human body and measurements of certain specific body fluids. The above technical means for evaluating the refreshing efficacy of aromatic essential oils are mainly based on the physical properties of the raw material of aromatic essential oils, preparation processes thereof, traditional experience and sensory judgment and experience. However, the technical means for evaluating the biological efficacies of aromatic essential oils lack the objective standard support at the biological level. Thus, the biological efficacies related to the refreshing efficacies of aromatic essential oils cannot be immobilized or quantified, leading to great limitations and uncertainties in the selection of raw materials of aromatic essential oils with a refreshing efficacy, formulation applications and efficacies promotion in industrial applications.

### SUMMARY

in view of this, the present disclosure provides a method for screening essential oils with a refreshing efficacy or their products. The method has characteristics of fast and accurate, data repeatable, method immobilized, standards objective and safe in use.

In order to achieve the above object, the following technical solution is provided in the present disclosure.

The present disclosure provides a method for screening essential oils with a refreshing efficacy or their products, comprising the steps of

treating brain neuron cells with essential oils or their products, determining concentrations of 5-hydroxy tryptamine, γ-aminobutyric acid, melatonin, glycine, dopamine, glutamic acid, norepinephrine and acetylcholine to obtain a concentration of neurotransmitter in a treatment group;

determining change rate of the concentration of neurotransmitter in the treatment group compared to a concentration of neurotransmitter in a blank control group according to the following formula:

$${V}_{C} = \frac{{C}_{1} - {C}_{0} }{{C}_{0} } ,$$

wherein, C₁ is the concentration of neurotransmitter in the treatment group, and C₀ is the concentration of neurotransmitter in the blank control group, and V_{c} is the change rate of the concentration of neurotransmitter in the treatment group compared to the concentration of neurotransmitter in the blank control group; and

respectively obtaining refreshing scores Xᵢ of the 5-hydroxy tryptamine, γ-aminobutyric acid, melatonin, glycine, dopamine, glutamic acid, norepinephrine and acetylcholine, which are shown as follows:

| | 5-hydroxy | γ-aminobutyric | | | | glutamic | | |
|---|---|---|---|---|---|---|---|---|
| Change rate V_{c} | tryptamine | acid | melatonin | glycine | dopamine | acid | norepinephrine | acetylcholine |
| ≤−44.4% | 10 | 10 | 10 | 10 | 1 | 1 | 1 | 1 |
| −44.4% < Vc ≤ −37.5% | 9 | 9 | 9 | 9 | 2 | 2 | 2 | 2 |
| −37.5% < Vc ≤ −28.6% | 8 | 8 | 8 | 8 | 3 | 3 | 3 | 3 |
| −28.6% < Vc ≤ −16.7% | 7 | 7 | 7 | 7 | 4 | 4 | 4 | 4 |
| −16.7% < V_{c} ≤ 0% | 6 | 6 | 6 | 6 | 5 | 5 | 5 | 5 |
| 0 < V_{c} ≤ 20% | 5 | 5 | 5 | 5 | 6 | 6 | 6 | 6 |
| 20% < V_{c} ≤ 40% | 4 | 4 | 4 | 4 | 7 | 7 | 7 | 7 |
| 40% < V_{c} ≤ 60% | 3 | 3 | 3 | 3 | 8 | 8 | 8 | 8 |
| 60% < V_{c} ≤ 80% | 2 | 2 | 2 | 2 | 9 | 9 | 9 | 9 |
| >80% | 1 | 1 | 1 | 1 | 10 | 10 | 10 | 10 |

determining a total refreshing score X of the essential oils or their products according to the following formula:

X=X₁+X₂+X₃+X₄+X₅+X₆+X₇+X₈,

wherein X is the total refreshing score, X₁ is the refreshing core of 5-hydroxy tryptamine, X₂ is the refreshing core of γ-aminobutyric acid, X₃ is the refreshing core of melatonin, X₄ is the refreshing core of glycine, X₅ is the refreshing core of dopamine, X₆ is the refreshing core of glutamic acid, X₇ is the refreshing core of norepinephrine, and X₈ is the refreshing core of acetylcholine;

where X≥44, the essential oils or their products are essential oils having a refreshing efficacy or their products; and

where X<44 the essential oils or their products are not essential oils having a refreshing efficacy or their products.

Preferably, mass percentage concentration of the essential oils or their products is 0.00005%-0.05%.

Preferably, mass percentage concentration of the essential oils is 0.0005%.

Preferably, the duration that the essential oils or their products treat the brain neuron cells is 5-60 min.

Preferably, the treatment duration is 30 min.

Preferably, the brain neuron cells treated with the essential oils or their products are brain neuron cells cultured for 5-15 days.

Preferably, the brain neuron cells are brain neuron cells cultured for 10 days.

Preferably, the culture medium for the culture is a horse serum culture solution, inoculation amount of the cells is 1×10⁴ cells/mL, culture temperature is 37° C., and culture condition is 5% CO₂.

Preferably, the brain neuron cells are brain neuron cells of a human body or brain neuron cells of an animal.

In the embodiments of the present disclosure, the brain neuron cells of an animal are brain neuron cells of murine.

In the embodiments of the present disclosure, the brain neuron cells of an animal are brain neuron cells of mouse.

The present disclosure provides a method for screening essential oils with a refreshing efficacy or their products. The method comprises: treating brain neuron cells with essential oils or their products, determining concentrations of 5-hydroxy tryptamine and the like to obtain a concentration of neurotransmitter in a treatment group; determining change rate V_{c} of the concentration of neurotransmitter in the treatment group compared to a concentration of neurotransmitter in a blank control group; respectively obtaining refreshing scores Xᵢ of each neurotransmitter according to V_{c}; respectively determining a total refreshing score X of the essential oils or their products according to the summation formula; where X≥44, the essential oils or their products are essential oils having a refreshing efficacy or their products. The present disclosure has at least one of the following advantages:

The present disclosure can quantitatively evaluate the refreshing efficacy of raw materials and products of aromatic essential oils from the in vitro biological cells and in their physiology and biology aspects.

The present disclosure can provide biological objective data of the refreshing efficacy of the aromatic essential oils and their products, and quantify the evaluation of the refreshing efficacy of the aromatic essential oils and their products.

The present disclosure can repeatedly verify the refreshing efficacy of the aromatic essential oils and their products.

The quantitative evaluation and verification of the refreshing efficacy of the aromatic essential oils and their products are both carried out in vitro and are easy to operate. At the same time, the in vitro evaluation method has a characteristic of reproducible immobilization.

The method for evaluating the refreshing efficacy of the aromatic essential oils and their products can effectively support the raw materials selection, formulation design and preparation and function detection of the aromatic essential oils with refreshing efficacy.

It can be seen that the method in the present disclosure can carry out biological in-vitro evaluation of the refreshing efficacy of the aromatic essential oils and their products, having characteristics of being fast and accurate, data repeatable, method immobilized, standards objective and safe in use.

The method of the present disclosure can accelerate the screening and evaluation process of the aromatic essential oils and their products, perform a fast evaluation on establishing a formulation of aromatic essential oils having a refreshing efficacy and their products, and provide users of the aromatic essential oils having a refreshing efficacy and their products with efficacy descriptions of the products, safety descriptions of the products, administration dosage descriptions of the product and method for using of the products

### BRIEF DESCRIPTION OF DRAWINGS

FIG. 1 shows the growth status of the brain neuron cells on the 10^{th} day.

FIGS. 2-4 respectively show the levels of dopamine, γ-aminobutyric acid and melatonin released from the neuron cells in the culture solution at 0-60 min after adding the mint peppermint Yakima essential oil in the brain neuron cells.

FIG. 5 shows the influence of lemongrass essential oil on the level of brain neuron cells neurotransmitter dopamine.

FIGS. 6-13 respectively show the influences of different aromatic essential oils on the levels of the 5-hydroxy tryptamine, γ-aminobutyric acid, acetylcholine, norepinephrine, glycine, glutamic acid, melatonin and dopamine that released from the neuron cells.

FIG. 14 shows the technological process of the present disclosure.

### DETAILED DESCRIPTION

The present disclosure provides a method for screening essential oils with a refreshing efficacy and their products is provided. One ordinary skilled in the art can learn from the contents of this document and appropriately improve the process parameters. It should be noted that all such alternatives and modifications are obvious to one ordinary skilled in the art and are considered to be included in the present disclosure. The methods and applications of the present disclosure have been described in preferred embodiments. It will be apparent that the related people may modify or amend and combine as appropriate the methods and applications described herein without departing form the contents of the present disclosure to implement and apply the techniques of the present disclosure.

In the following embodiments, % is the mass percentage without special illustration.

In the following embodiments, the level of each neurotransmitter is determined with a kit purchased from the market, which comprises a color developing agent A and a color developing agent B.

The essential oils or neurons cells used in the method for screening essential oils with a refreshing efficacy or their products in the present disclosure may all purchased from the market.

The present disclosure will be further illustrated in conjunction with the embodiments hereinafter.

### EXAMPLE 1

### Culture of the Brain Neuron Cells

1. Culture of Primary Mouse Brain Neuron Cells

1-day newly born mice were selected. The neurons cells in the brain of the mice were separated and extracted, and were dispersed into Neurobasal Medium (Thermo Fisher, Cat.# 21103-049) culture solution containing horse serum. Then the resultant was cultured in a cell incubator at 37° C., having a culture condition of air:carbon dioxide=95:5. The culture solution of brain neuron cells was changed every other day. The growth status of the brain neuron cells on the tenth day of culture was shown in FIG. 1.

2. Culture of Primary Human Brain Neuron Cells

Human brain neuron cells were commercially purchased. The cells were disposed in Neurobasal Medium (Thermo Fisher, Cat.# 21103-049) culture solution containing horse serum. Then the resultant was cultured in a cell incubator at 37° C., having a culture condition of air:carbon dioxide=95:5. The culture solution of brain neuron cells was changed every other day. The growth status of the brain neuron cells on the tenth day of culture was similar to that showed in FIG. 1.

### EXAMPLE 2

### Influence of Mint Peppermint Yakima Essential Oil on the Release Level of 3 Neurotransmitters in Brain Neuron Cells

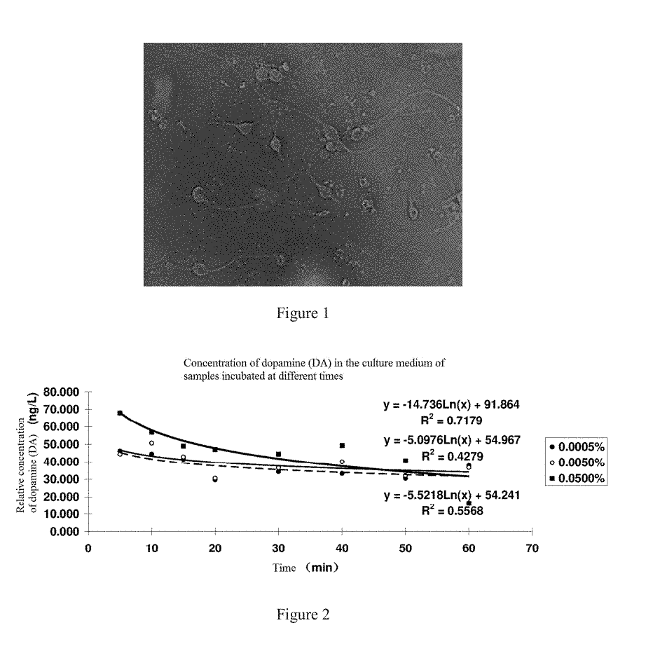

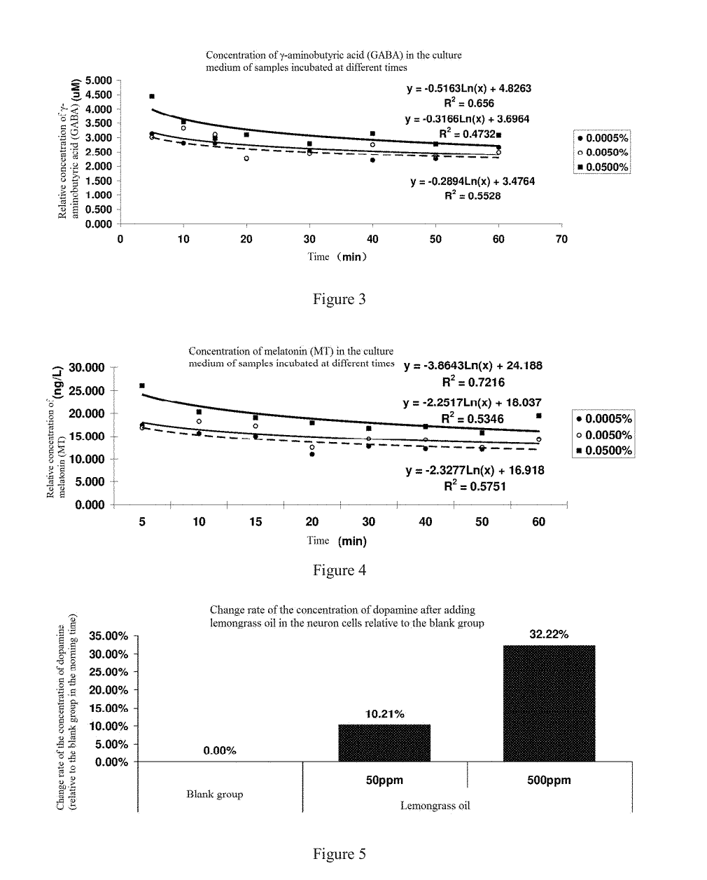

Influence of mint peppermint Yakima essential oil on the release level of the neurotransmitter in the brain neuron cells is a function of time. The mint peppermint Yakima essential oil was added to the brain neuron cells culture system on the tenth day of culture, such that the final concentration of the mint peppermint Yakima essential oil in the brain neuron cells culture system was 0.0005%, 0.0050% and 0.0500%. Levels of dopamine, γ-aminobutyric acid and melatonin released from the neurons cells in the culture solution at 0-60 min after adding the mint peppermint Yakima essential oil in the brain neuron cells were measured, in the examples of the present disclosure, the neurotransmitter samples were collected at the 5^{th} minute, the 10^{th} minute, the 15^{th} minute, the 20^{th} minute, the 30^{th} minute, the 40^{th} minute, the 50^{th} minute and the 60^{th} minute. The results were shown in FIGS. 2-4.

The results showed that influence of the mint peppermint Yakima essential oil on releasing neurotransmitters such as dopamine, γ-aminobutyric acid and melatonin from the brain neuron cells became apparent at the 5^{th} min after the sample was added, and the process lasted for 60 min. This data indicated that there was a certain linear relationship between the release of the neurotransmitter in the brain neuron cells treated with the aromatic essential oils and the sample collecting time from the 5^{th} minute to the 30^{th} minute.

### EXAMPLE 3

### Influence of Mint Peppermint Yakima Essential Oil on the Release Level of Numerous Neurotransmitters in Brain Neuron Cells

At the 30^{th} minute, the influence of mint Peppermint Yakima Oil at different concentrations on the release level of different neurotransmitters in brain neuron cells in the neuron culture system was shown in Table 1. The neurotransmitters involved in the present disclosure comprised DOPAMINE, 5-HYDROXY TRYPTAMINE, GAMMA AMINOBUTYRIC ACID, ACETYLCHOLINE, NORADRENALINE, GLYCINE, GLUTAMINE and MELATONIN.

TABLE 1
Influence of mint peppermint Yakima essential oil on the release level of different neurotransmitters in brain neuron cells

| | Concen- | 5-hydroxy | γ- | | | | | | |
|---|---|---|---|---|---|---|---|---|---|
| | tration | tryptamine | aminobutyric | glycine | melatonin | acetylcholine | dopamine | glutamic | norepinephrine |
| Treatment | (%) | (μM) | acid (ng/L) | (ng/L) | (ng/L) | (pM) | (ng/L) | acid (μM) | (ng/L) |
| blank | — | 278.7 ± 42.3 | 6.3 ± 0.6 | 72.5 ± 8.1 | 25.5 ± 1.7 | 211.3 ± 14.3 | 81.8 ± 6.7 | 13.6 ± 1.0 | 73.8 ± 14.6 |
| Mint | 0.050% | 271.3 ± 11.7 | 6.1 ± 0.8 | 60.9 ± 4.9 | 32.0 ± 4.6 | 206.3 ± 4.1 | 82.6 ± 8.8 | 11.3 ± 1.9 | 84.7 ± 12.0 |
| peppermint | | | | | | | | | |
| Yakima | | | | | | | | | |
| essential oil | | | | | | | | | |
| Mint | 0.005% | 189.9 ± 14.4 | 4.7 ± 0.8 | 51.9 ± 5.8 | 29.4 ± 3.7 | 175.0 ± 22.1 | 83.2 ± 15.9 | 10.8 ± 1.8 | 76.3 ± 10.1 |
| peppermint | | | | | | | | | |
| Yakima | | | | | | | | | |
| essential oil | | | | | | | | | |

The above experimental results showed that at a certain concentration, the mint peppermint Yakima essential oil promotes the release of melatonin, dopamine and norepinephrine, and inhibits the release of 5-hydroxy tryptamine, γ-aminobutyric acid, glycine, acetylcholine and glutamic acid. In addition, the experimental result showed that there is a certain dose-effect relationship between different dosages of mint peppermint essential oil and promotion or inhibition of the release of different neurotransmitters.

### EXAMPLE 4

### Influence of Lemongrass Oil on Level of Neurotransmitter Dopamine in Brain Neuron Cells

The present example related to analysis on influence of lemongrass oil on level of neurotransmitter dopamine in brain neuron cells and the refreshing efficacy.

Test method: The lemongrass oil was added to the culture system of the brain neuron cells on the tenth day of culture, such that the final concentration of the lemongrass oil in the brain neuron cells culture system was 0%, 0.00005% and 0.0005%. Level of neurotransmitter dopamine released from the neurons cells in the culture solution at the 30^{th} min after adding the essential oil in the brain neuron cells was measured.

The test results were shown in FIG. 5. The experimental data showed that different concentrations of lemongrass oil increased the level of dopamine in brain neuron cells. Dopamine is closely related to the transmission of information such as feeling, excitement, and memory of human. Thus, in the present disclosure, it can be concluded from the data obtained in this measurement that at a concentration of 500 ppm, excitatory stimulation effect of the lemongrass oil on the brain neuron cells increased by 32.22%. Formulating the corresponding products at this concentration can obtain a better refreshing function on human body.

### EXAMPLE 5

### Method for Screening the Essential Oils with a Refreshing Efficacy

Influences of different aromatic essential oils on the level of numerous neurotransmitters in brain neuron cells were studied in this example. The concentrations of the aromatic essential oils in this example were all 0.0005%.

Test method: Essential oils were added to the culture system of the brain neuron cells on the tenth day of culture, such that the final concentration of the essential oils in the brain neuron cells culture system was 0.0005%. Level of neurotransmitter released from the neurons cells in the culture solution at the 30^{th} rain after adding the essential oils in the brain neuron cells was measured.

The neurotransmitters in this example included DOPAMINE, 5-HYDROXY TRYPTAMINE, GAMMA AMINOBUTYRIC ACID, ACETYLCHOLINE, NORADRENALINE, GLYCINE, GLUTAMINE and MELATONIN.

The aromatic essential oils in this example included HOHE, ROSEMARY OIL, BASIL OIL, LEMONGRASS OIL, YLANG III MADAGASCAR EO, MINT PEPPERMINT YAKIMA OIL, LEMON OIL ITALY, and EUCALYPTUS GLOBULUS OIL.

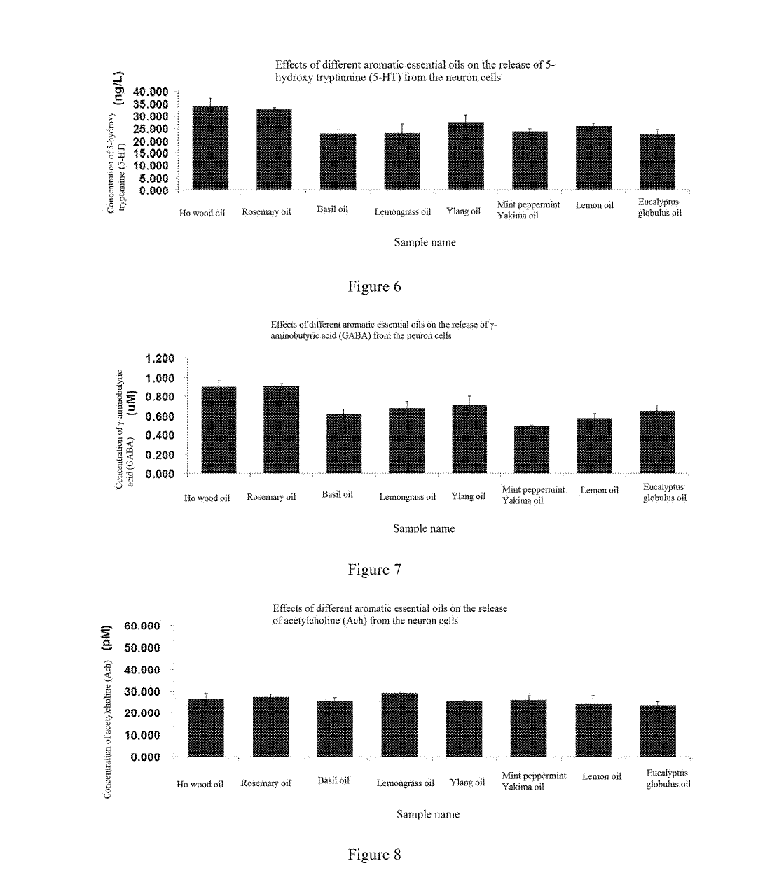

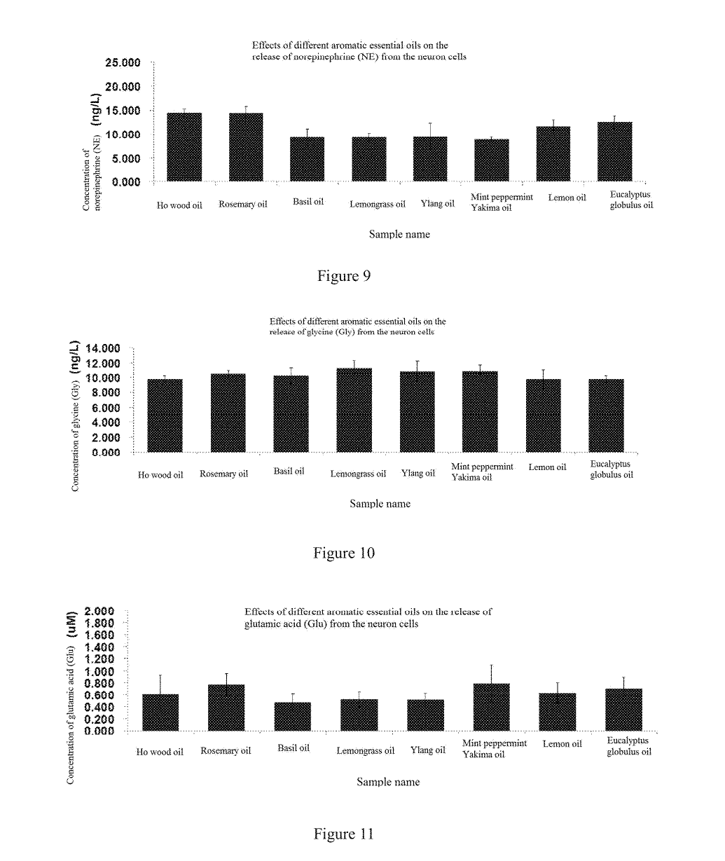

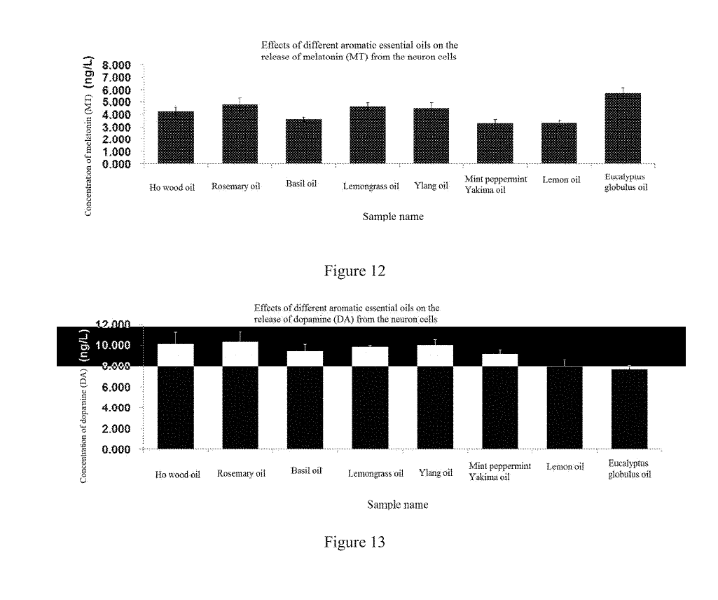

Test results of influences of different aromatic essential oils on the level of numerous neurotransmitters after treating the brain neuron cells were shown in FIGS. 6-13.

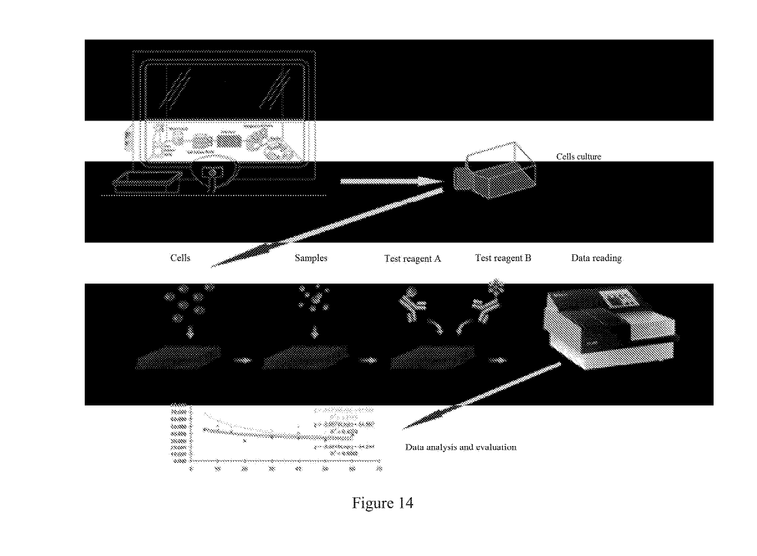

According to the above test results, the following evaluation system was used to calculate the refreshing scores of each aromatic essential oil. Technological process diagram was shown in FIG. 14. Standards of the evaluation system of the refreshing functions were shown in Table 2, and the calculated results were shown in Table 3.

TABLE 2
Standards of the evaluation system of the refreshing functions of the aromatic essential oils on brain neuron cells

| | | γ- | | | | | | |
|---|---|---|---|---|---|---|---|---|
| Change rate V_{c} of | 5-hydroxy | aminobutyric | | | | glutamic | norepine | |
| the measured value | tryptamine | acid | melatonin | glycine | dopamine | acid | phrine | acetylcholine |
| and the blank value | (5-HT) | (GABA) | (MT) | (Gly) | (DA) | (Glu) | (NE) | (Ach) |
| ≤−44.4% | 10 | 10 | 10 | 10 | 1 | 1 | 1 | 1 |
| −44.4% < Vc ≤ −37.5% | 9 | 9 | 9 | 9 | 2 | 2 | 2 | 2 |
| −37.5% < Vc ≤ −28.6% | 8 | 8 | 8 | 8 | 3 | 3 | 3 | 3 |
| −28.6% < Vc ≤ −16.7% | 7 | 7 | 7 | 7 | 4 | 4 | 4 | 4 |
| −16.7% < V_{c} ≤ 0% | 6 | 6 | 6 | 6 | 5 | 5 | 5 | 5 |
| 0 < V_{c} ≤ 20% | 5 | 5 | 5 | 5 | 6 | 6 | 6 | 6 |
| 20% < V_{c} ≤ 40% | 4 | 4 | 4 | 4 | 7 | 7 | 7 | 7 |
| 40% < V_{c} ≤ 60% | 3 | 3 | 3 | 3 | 8 | 8 | 8 | 8 |
| 60% < V_{c} ≤ 80% | 2 | 2 | 2 | 2 | 9 | 9 | 9 | 9 |
| >80% | 1 | 1 | 1 | 1 | 10 | 10 | 10 | 10 |

Note:
The higher the score is, the higher the concentration of excitatory neurotransmitter is or the lower the concentration of the inhibitory neurotransmitter is.

TABLE 3
Refreshing scores of the aromatic essential oils on brain neuron cells

| | | Concentration | | | | | | | | | |
|---|---|---|---|---|---|---|---|---|---|---|---|
| | | of | | | | | | | | | |
| | Name of the | the sample | 5- | | | | | | | | Total |
| Experiment | sample | (ppm) | HT | GABA | MT | Gly | DA | Glu | NE | Ach | score |
| Experiment 1 | lemongrass oil | 50 | 1 | 1 | 5 | 1 | 10 | 7 | 7 | 10 | 42 |
| | mint peppermint | 50 | 5 | 5 | 5 | 6 | 6 | 7 | 5 | 6 | 45 |
| | Yakima oil | | | | | | | | | | |
| | ho wood oil | 50 | 6 | 4 | 7 | 4 | 7 | 6 | 5 | 5 | 44 |
| | rosemary oil | 50 | 5 | 5 | 6 | 4 | 7 | 6 | 5 | 5 | 43 |
| | basil oil | 50 | 6 | 4 | 6 | 3 | 7 | 6 | 5 | 6 | 43 |
| | ylang oil | 50 | 5 | 2 | 6 | 3 | 7 | 6 | 6 | 5 | 40 |
| | lemon oil | 50 | 6 | 5 | 6 | 4 | 6 | 5 | 5 | 5 | 42 |
| | cucalyptus | 50 | 5 | 5 | 6 | 4 | 6 | 6 | 5 | 5 | 42 |
| | globulus oil | | | | | | | | | | |
| Experiment 2 | Lemongrass oil | 1 | 6 | 6 | 6 | 6 | 5 | 5 | 6 | 5 | 45 |
| | | 10 | 5 | 6 | 6 | 6 | 5 | 5 | 6 | 5 | 44 |
| | Mint peppermint | 0.1 | 6 | 6 | 6 | 6 | 5 | 5 | 5 | 5 | 44 |
| | Yakima oil | 0.5 | 5 | 5 | 5 | 5 | 6 | 6 | 7 | 6 | 45 |
| | | 1 | 5 | 5 | 6 | 5 | 5 | 6 | 7 | 6 | 45 |
| | | 5 | 5 | 6 | 6 | 6 | 5 | 5 | 6 | 6 | 45 |
| | | 10 | 5 | 6 | 6 | 6 | 6 | 6 | 6 | 6 | 47 |
| | | 50 | 4 | 4 | 4 | 4 | 7 | 7 | 7 | 7 | 44 |
| | | 100 | 4 | 4 | 4 | 4 | 7 | 7 | 8 | 7 | 45 |
| Experiment 3 | *Es32 | 50 | 3 | 1 | 2 | 1 | 1 | 7 | 6 | 7 | 28 |
| | *Es10 | 50 | 9 | 6 | 5 | 7 | 7 | 7 | 6 | 4 | 51 |

*Note: Es10: Mint peppermint Yakima oil 12% + lemongrass oil 7% + eucalyptus citriodora essential oil 4% + grapefruit essential oil 5% + bergamot essential oil 8% + basil essential oil 6% + myrrh essential oil 8% + sweet almond essential oil 50%; and Es32: bitter orange essential oil 6% + super lavender essential oil 8% + ylang essential oil 4% + geranium essential oil 4% + cypress essential oil 3% + cymbopogon martini essential oil 4% + Roman chamomile essential oil 6% + orange blossom essential oil 7% + benzoin essential oil 8% + sweet almond oil 50%.

In the present disclosure, a score of 44 were regarded as a boundary. The higher the total score was, the better the refreshing efficacy of the sample was. Data in Table 3 showed that total scores of the release level of the neurotransmitters in brain neuron cells treated with mint peppermint Yakima oil and lemongrass oil at some concentrations were more than 44 points. This indicated that at corresponding concentrations, these samples had a relatively good refreshing efficacy. However, total scores of the release level of the neurotransmitters in brain neuron cells treated with other essential oil samples at corresponding concentrations did not exceed 44 points. This indicated that at the corresponding concentrations, these samples did not have a relatively good refreshing efficacy. In Experiment 3. the samples Es32 and Es10 were respectively a negative control and a positive control. Total scores of the release level of the neurotransmitter in brain neuron cells treated with Es32 were 28, and total scores of the release level of the neurotransmitter in brain neuron cells treated with Es10 were 51. This indicated that this evaluation method can be used to obtain a judgment consistent with the control sample on the refreshing efficacy.

The above descriptions are only the preferred embodiments of the present disclosure. It should be noted that one ordinary skilled in the art can make a number of modifications and refinements without departing from the principles of the disclosure, and such modifications and refinements are also considered to be within the protection scope of the disclosure.

1. A method for screening essential oils with a refreshing efficacy or their products, comprising the steps of
treating brain neuron cells with essential oils or their products, determining concentrations of 5-hydroxy tryptamine, γ-aminobutyric acid, melatonin, glycine, dopamine, glutamic acid, norepinephrine and acetylcholine to obtain a concentration of neurotransmitter in a treatment group;
determining change rate of the concentration of neurotransmitter in the treatment group compared to a concentration of neurotransmitter in a blank control group according to the following formula:
$${V}_{c} = \frac{{C}_{1} - {C}_{0 } }{{C}_{0 } } ,$$
wherein, C₁ is the concentration of neurotransmitter in the treatment group, and C₀ is the concentration of neurotransmitter in the blank control group, and V_{c} is the change rate of the concentration of neurotransmitter in the treatment group compared to the concentration of neurotransmitter in the blank control group; and
respectively obtaining refreshing scores Xᵢ of the 5-hydroxy tryptamine, γ-aminobutyric acid, melatonin, glycine, dopamine, glutamic acid, norepinephrine and acetylcholine, which are shown as follows:

| | | γ- | | | | | | |
|---|---|---|---|---|---|---|---|---|
| | 5-hydroxy | aminobutyric | | | | glutamic | norepine | |
| Change rate V_{c} | tryptamine | acid | melatonin | glycine | dopamine | acid | phrine | acetylcholine |
| ≤−44.4% | 10 | 10 | 10 | 10 | 1 | 1 | 1 | 1 |
| −44.4% < Vc ≤ −37.5% | 9 | 9 | 9 | 9 | 2 | 2 | 2 | 2 |
| −37.5% < Vc ≤ −28.6% | 8 | 8 | 8 | 8 | 3 | 3 | 3 | 3 |
| −28.6% < Vc ≤ −16.7% | 7 | 7 | 7 | 7 | 4 | 4 | 4 | 4 |
| −16.7% < V_{c} ≤ 0% | 6 | 6 | 6 | 6 | 5 | 5 | 5 | 5 |
| 0 < V_{c} ≤ 20% | 5 | 5 | 5 | 5 | 6 | 6 | 6 | 6 |
| 20% < V_{c} ≤ 40% | 4 | 4 | 4 | 4 | 7 | 7 | 7 | 7 |
| 40% < V_{c} ≤ 60% | 3 | 3 | 3 | 3 | 8 | 8 | 8 | 8 |
| 60% < V_{c} ≤ 80% | 2 | 2 | 2 | 2 | 9 | 9 | 9 | 9 |
| >80% | 1 | 1 | 1 | 1 | 10 | 10 | 10 | 10 |

determining a total refreshing score X of the essential oils or their products according to the following formula:
X=X₁+X₂+X₃+X₄+X₅+X₆+X₇+X₈,
wherein X is the total refreshing score, X₁ is the refreshing core of 5-hydroxy tryptamine, X₂ is the refreshing core of γ-aminobutyric acid, X₃ is the refreshing core of melatonin, X₄ is the refreshing core of glycine, X₅ is the refreshing core of dopamine, X₆ is the refreshing core of glutamic acid, X₇ is the refreshing core of norepinephrine, and X₈ is the refreshing core of acetylcholine.

2. The method according to claim 1, wherein mass percentage concentration of the essential oils or their products is 0.00005%-0.05%.

3. The method according to claim 2, wherein the mass percentage concentration of the essential oils is 0.0005%.

4. The method according to claim 1, wherein the treatment duration is 5-60 min.

5. The method according to claim 4, wherein the treatment duration is 30 min.

6. The method according to claim 1, wherein the brain neuron cells are brain neuron cells cultured for 5-15 days.

7. The method according to claim 6, wherein the brain neuron cells are brain neuron cells cultured for 10 days.

8. The method according to claim 6, wherein culture medium for the culture is a horse serum culture solution, inoculation amount of the cells is 1×10⁴ cells/mL, culture temperature is 37° C., and culture condition is 5% CO₂.

9. The method according to claim 1, wherein the brain neuron cells are brain neuron cells of a human body or brain neuron cells of an animal.

10. The method according to claim 9, wherein the brain neuron cells of an animal are brain neuron cells of murine.
